(19) United States
(12) Patent Application Publication
(10) Pub. No.: US 2012/0022506 A1
(43) Pub. Date: Jan. 26, 2012

(54) Drug delivery device with active iris

(76) Inventors: Matthew J.A. Rickard, Yorba Linda, CA (US); Robert J. Sanchez, JR., Oceanside, CA (US); Bruno Dacquay, Irvine, CA (US)

(21) Appl. No.: 13/113,800

(22) Filed: May 23, 2011

Related U.S. Application Data
(60) Provisional application No. 61/366,009, filed on Jul. 20, 2010.

(51) Int. Cl. A61F 9/00

(52) U.S. Cl. 604/891.1; 604/66

(57) ABSTRACT

A device implantable into an eye of a patient for treatment of glaucoma. The device has an implantable dispenser. The dispenser includes an implantable reservoir configured to store a therapeutic agent. Additionally, the dispenser includes an implantable reservoir sensor configured to measure a pressure within the reservoir. The device also has an implantable processor coupled to the implantable reservoir sensor and configured to receive the measurement of the pressure within the reservoir and determine a dosage of therapeutic agent based on the measurement of the pressure within the reservoir. Furthermore, the implantable dispenser is configured to release the dosage of the therapeutic agent at a selectively variable rate from the implantable reservoir into the eye.

This application claims priority to U.S. Provisional Patent Application Ser. No. 61/366,009 filed on Jul. 20, 2010.

### BACKGROUND OF THE INVENTION

The present disclosure relates generally to an implantable variable drug delivery system and more specifically to an implantable variable drug delivery system with an active iris for treatment of eye disorders such as glaucoma.

The eye's ciliary body epithelium constantly produces aqueous humor, the clear fluid that fills the anterior chamber of the eye (the space between the cornea and iris). The aqueous humor flows out of the anterior chamber through the uveoscleral pathways, a complex drainage system. The delicate balance between the production and drainage of aqueous humor determines the eye's IOP.

Glaucoma, a group of eye diseases affecting the retina and optic nerve, is one of the leading causes of blindness worldwide. Glaucoma results when the IOP increases to pressures above normal for prolonged periods of time. IOP can increase due to an imbalance of the production of aqueous humor and the drainage of the aqueous humor. Left untreated, an elevated IOP causes irreversible damage to the optic nerve and retinal fibers resulting in a progressive, permanent loss of vision.

Open angle (also called chronic open angle or primary open angle) is the most common type of glaucoma. With this type, even though the anterior structures of the eye appear normal, aqueous fluid builds within the anterior chamber, causing the IOP to become elevated. Left untreated, this may result in permanent damage of the optic nerve and retina.

Patients are typically prescribed a recommended drug dosage in the form of eye drops to treat eye disorders such as glaucoma. These dosages are based on the evaluation results taken at the office visit. However, because the symptoms requiring treatment can change or vary over time, the prescribed dosage may not be the most effective dosage, or may exceed a recommended dosage for the particular symptom. Prescribed dosage levels typically change only when a patient makes a new office visit to a health care provider for an additional evaluation. What is needed is a system and method that effectively varies therapeutic agent dosage levels in the eye based on variable eye conditions or based on recommended dosage levels determined by a health care provider.

The systems, devices, and methods disclosed herein overcome at least one of the shortcomings in the prior art.

### SUMMARY OF THE INVENTION

In one exemplary aspect, the present disclosure is directed to a device implanted into an eye of a patient for treatment of glaucoma. The device has an implantable dispenser. The dispenser includes an implantable reservoir configured to store a therapeutic agent. Additionally, the dispenser includes an implantable reservoir sensor configured to measure a pressure within the reservoir. The device also has an implantable processor coupled to the implantable reservoir sensor and configured to receive the measurement of the pressure within the reservoir and determine a dosage of therapeutic agent based on the measurement of the pressure within the reservoir. Furthermore, the implantable dispenser is configured to release the dosage of the therapeutic agent at a selectively variable rate from the implantable reservoir into the eye.

In another exemplary aspect, the present disclosure is directed to a method for delivering a dosage of a therapeutic agent into an eye. The method includes implanting a system into the eye. The system has an implantable dispenser. The implantable dispenser includes an implantable reservoir configured to store the therapeutic agent. Also, the implantable dispenser includes an implantable reservoir sensor configured to measure a pressure within the implantable reservoir. The system also has an implantable processor coupled to the implantable reservoir sensor and configured to receive the measurement of the pressure within the implantable reservoir. Furthermore, the implantable dispenser is configured to release the dosage of the therapeutic agent stored in the implantable reservoir into the eye based on the measurement of the pressure within the implantable reservoir. Additionally, the method includes measuring the pressure within the implantable reservoir with the implantable reservoir sensor. Also, the method includes determining the dosage of the therapeutic agent with the implantable processor by using the measured pressure within the implantable reservoir to determine the dosage. In addition, the method includes actuating the dispenser to dispense the dosage of the therapeutic agent at a selectively variable rate from the implantable reservoir into the eye in response to a signal from the implantable processor.

In yet another exemplary aspect, the present disclosure is directed to a device implanted into an eye of a patient for treatment of glaucoma. The device has an implantable dispenser. The implantable dispenser includes an implantable reservoir configured to store a therapeutic agent. Additionally, the implantable sensor has an implantable reservoir sensor configured to measure a pressure within the reservoir. The device further includes an implantable intraocular pressure sensor configured to measure an intraocular pressure of the eye. Also, the device has an implantable processor coupled to the implantable reservoir sensor and configured to receive the measurement of the pressure within the reservoir and the measurement of the intraocular pressure. The implantable processor further configured to determine a dosage of the therapeutic agent based on the measurement of the pressure within the reservoir and the measurement of the intraocular pressure. Furthermore, the implantable dispenser is configured to release the dosage of the therapeutic agent at a selectively variable rate from the implantable reservoir into the eye.

These and other aspects, forms, objects, features, and benefits of the present disclosure will become apparent from the following detailed drawings and description.

### BRIEF DESCRIPTION OF THE DRAWINGS

The accompanying drawings, which are incorporated in and constitute a part of the specification, illustrate embodiments of the present disclosure. Together with a general description of the present disclosure given above, and the detailed description given below, the accompanying drawings serve to exemplify the embodiments of the present disclosure.

FIG. 1 is a block diagram of an exemplary implantable variable drug delivery system according to one aspect of the present disclosure.

FIG. 2A is an illustration of an exemplary dispenser of the implantable variable drug delivery system of FIG. 1 being filled with a therapeutic agent.

FIG. 2B is an illustration of the exemplary dispenser of FIG. 2B dispensing the therapeutic agent.

FIGS. 3A-3C are illustrations of an exemplary shutter assembly of the implantable variable drug delivery system of FIG. 1 having an active iris to control the flow rate of a therapeutic agent being dispensed by the shutter assembly.

FIG. 4 is an illustration of a perspective view of a patient's eye with an implantable portion of the variable drug delivery system of FIG. 1 implanted within the patient's eye.

FIGS. 5A-5C are illustrations of another exemplary shutter assembly having an active iris to control the flow rate of a therapeutic agent being dispensed by the shutter assembly.

FIGS. 6A-6C are illustrations of another exemplary shutter assembly having an active iris to control the flow rate of a therapeutic agent being dispensed by the shutter assembly.

FIG. 7A is an illustration of another exemplary dispenser being filled with a therapeutic agent.

FIG. 7B is an illustration of the exemplary dispenser of FIG. 7B dispensing the therapeutic agent.

FIG. 8 is an exemplary flow diagram showing steps for determining the drug dosage delivered into the patient's eye using the implantable variable drug delivery system of FIG. 1.

### DETAILED DESCRIPTION OF THE INVENTION

The present disclosure relates generally to the field of ophthalmic surgery, and more particularly to an implantable variable drug system and more specifically to an implantable variable drug delivery system with an active iris for treatment of eye disorders such as glaucoma. For the purposes of promoting an understanding of the principles of the present disclosure, reference will now be made to embodiments or examples illustrated in the drawings, and specific language will be used to describe these examples. It will nevertheless be understood that no limitation of the scope of the present disclosure is thereby intended. Any alteration and further modifications in the described embodiments, and any further applications of the principles of the present disclosure as described herein are contemplated as would normally occur to one skilled in the art to which the disclosure relates.

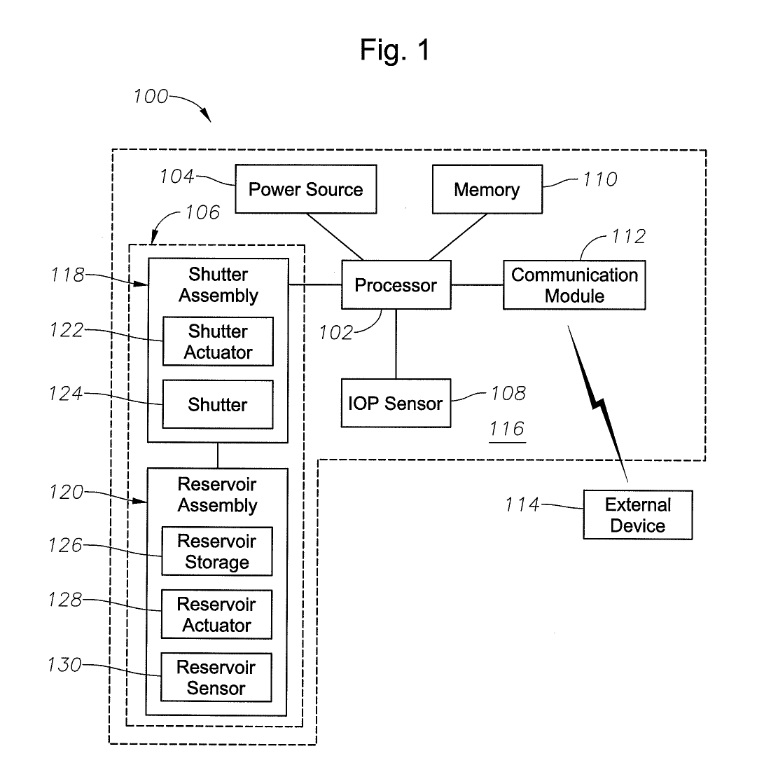

FIG. 1 is a schematic block diagram of an exemplary implantable variable drug delivery system according to one aspect of the present disclosure. The exemplary implantable drug delivery system 100 includes processor 102, power source 104, dispenser 106, IOP sensor 108, memory 110, communication module 112, and/or external device 114.

Processor 102 controls the operating functions of the implantable system 100 and may be an integrated circuit with power, input, and output pins capable of performing logic functions. In various embodiments, processor 102 is a targeted device controller. In such a case, processor 102 performs specific control functions targeted to a specific device or component, such as a power source 104, dispenser 106, IOP sensor 108, memory 110, and/or communication module 112.

In other embodiments, processor 102 is a microprocessor. In such a case, processor 102 is programmable so that it can function to control one or more of the components of system 100. In other cases, processor 102 is not a programmable microprocessor, but instead is a special purpose controller configured to control different components that perform different functions.

Power source 104 may be a rechargeable battery, such as a lithium ion or lithium polymer battery, although other types of batteries may be employed. Additionally, it is contemplated that power source 104 can be any type of power cell that is appropriate for implantation into the patient's body. In some embodiments, power source 104 is controllable by processor 102 to provide power to all the elements making up system 100. In other words, power source 104 may provide power to any component of system 100 including, but not limited to processor 102, dispenser 106, IOP sensor 108, memory 110, and/or communication module 112. In other embodiments, some and/or all components of system 100 have their own independent power source. In some examples, power source 102 is configured to be recharged via an RFID (radio-frequency identification) link or other type of magnetic coupling, or inductive coupling.

Dispenser 106 is also coupled to processor 102. Specifically, dispenser 106 stores and administers a therapeutic agent or drug. Through dispenser 106, system 100 provides the ability to deliver variable dosages that treat a patient's eye disorders such as glaucoma. Thus, dispenser 106 is operable to deliver a therapeutic agent into a patient's eye to treat a disorder.

To accomplish this, dispenser 106 has a shutter assembly 118 and a reservoir assembly 120. The shutter assembly 118 has a shutter actuator 122 and a shutter 124. Shutter actuator 122 rotates, translates, or otherwise moves shutter 124 to release a therapeutic agent from dispenser 106. In that regard, the movement of shutter 124 defines a variable sized opening for the dispensing of a therapeutic agent. Because the size of the opening varies, the opening is referred to herein as an active iris. The active iris allows the therapeutic agent dispensing rate to vary by increasing or decreasing the cross sectional area of the active iris. Shutter assembly 118 is discussed in further details below.

Reservoir assembly 120 includes reservoir storage 126, reservoir actuator 128, and reservoir sensor 130. Reservoir storage 126 stores one or more therapeutic agents that are administered by system 100. The therapeutic agent may be, for example, a solid, liquid, granule, and/or soluble agent. In one example, the reservoir storage 126 is compartmentalized such that more than one therapeutic agent can be stored in two or more compartments of the reservoir. As such, system 100 is configured to deliver one or more therapeutic agents.

Furthermore, dispenser 106 has an inlet port that is in fluid communication with the reservoir storage 126. The inlet port enables in vivo filling and/or refilling of a therapeutic agent for system 100. It is further contemplated that the reservoir storage 126 may be refilled with a therapeutic agent that is either substantially the same or substantially different than the previous therapeutic agent.

Reservoir actuator 128 is an actuating mechanism that causes the release of a therapeutic agent stored in reservoir storage 126. In that regard, reservoir actuator 128 is a mechanism that moves a therapeutic agent from and/or through reservoir storage 126 to the shutter assembly 118 for dispensing. In the example shown, reservoir actuator 128 moves the therapeutic agent by applying compressive and/or pressure forces to the therapeutic agent. In one embodiment, reservoir actuator 128 works in combination with shutter actuator 122 to administer or dispense the therapeutic agent. For example, upon actuation of shutter actuator 122 to displace the shutter 124 and open the active iris, the reservoir actuator 128 releases a stored therapeutic agent such that the therapeutic agent is dispensed by shutter assembly 118.

Reservoir assembly 120 further includes reservoir sensor 130. Reservoir sensor 130 measures the pressure within reservoir storage 126. In other words, reservoir sensor 130 measures the storage pressure being applied against a therapeutic agent stored in reservoir storage 126. As will be discussed in greater detail below, measuring the storage pressure allows system 100 to accurately determine the flow rate for a therapeutic agent through shutter assembly 118.

Also coupled to processor 102 is IOP sensor 108. The IOP sensor 108 configured to measure IOP in a patient's eye. Depending on the embodiment, IOP sensor 108 includes one or more sensors. For example, IOP senor 108 comprises a first pressure sensor P1 and a second pressure sensor P2. Pressure sensor P1 can be located either within an anterior chamber of a patient's eye or in fluidic communication with the anterior chamber. As such, pressure sensor P1 is operable to measure a pressure in the anterior chamber of a patient's eye.

Moreover, pressure sensor P2 is positioned adjacent to or within the patient's eye and is operable to measure an atmospheric pressure. For example, pressure sensor P2 may be implanted in the eye under the conjunctiva, such that it measures atmospheric pressure. In additional examples, pressure sensor P2 is implanted in a subconjunctival space of the patient's eye. Regardless of location, pressure sensor P2 is operable to measure atmospheric pressure in the vicinity of the eye.

Based on the readings from pressure sensors P1 and P2 the processor 102 may determine a patient's IOP. For purposes of this disclosure, IOP is measured as the difference between the absolute pressure in the eye (e.g. measurement taken by P1) and atmospheric pressure (e.g. measurement taken by P2). Pressure readings can be taken by P1 and P2 based over any time interval. For example, in some embodiments, the pressure sensors P1 and P2 are programmed to continuously measure pressure, thereby providing real-time accuracy of the patient's IOP. In other embodiments, pressure readings are taken by P1 and P2 at pre-established time intervals. For example, readings may be taken every minute, hourly, daily, etc. Regardless of the frequency of the pressure readings, the patient's IOP can be calculated based on the difference between the pressure readings of P1 and P2.

As described above, the pressure readings of P1 and P2 can be used to calculate the patient's current IOP. They can also be used to calculate the patient's average IOP over a given time period. For example, the pressure readings of P1 and P2 can be used to calculate the patient's IOP for a given time of day and/or week. In other words, it is contemplated that system 100 can use the pressure readings of P1 and P2 to determine the patient's IOP based on any desired interval.

It is contemplated that pressure sensors P1 and P2 can be any type of pressure sensor suitable for implantation in the eye. Furthermore, pressure sensors P1 and P2 can be the same type of pressure sensor, or may be different types. Moreover, although IOP sensor 108 has been discussed as comprising two pressure sensors (e.g. P1 and P2) it is contemplated that a patient's IOP may be determined by using a single pressure sensor or using three or more pressure sensors. Accordingly, no limitation to the number of or type of pressure sensors is implied by the present disclosure.

Referring to FIG. 1, memory 110 is any type of suitable storage memory including, but not limited to flash memory, solid state memory, organic memory, inorganic memory, and others. Memory 110 interfaces with processor 102. Thus, processor 102 can write to and read data from memory 110.

In some examples, memory 110 is operable to store dosage parameters, or logic such as executable code. In that regard, memory 110 can store programming data (e.g. dosage parameters) accessible by processor 102 that enables the processor to determine the proper dosage of therapeutic agent to deliver to a patient. In other words, based on the data and code stored in memory 110, processor 102 determines the proper dosage for a patient and subsequently causes dispenser 106 to deliver the drug to the patient. In some examples, processor 102 is hard coded or programmed directly with such dosage parameters such that the processor can determine the proper dosage without accessing memory 110.

The memory 110 is also configured to store pressure readings of P1 and P2 as well as the pressure reading from reservoir sensor 130. For example, processor 102 receives data from the IOP sensor 108 and reservoir sensor 130 and subsequently writes the data to memory 110. In this manner, a series of IOP readings and reservoir pressure readings can be stored in memory 110. Processor 102 is also capable of performing other basic memory functions, such as erasing or overwriting memory 110, detecting when memory 110 is full, and other common functions associated with managing memory.

The communication module 112 in FIG. 1 is operable to transmit and receive a number of different types of data transmission, or signals to external systems. For example, as shown in FIG. 1, communication module 112 can communicate with external device 114. Communication module 112 is operable to transmit and/or receive any data relating to system 100. For example in some embodiments, communication module 112 is operable to transmit and receive data relating to the measured pressure readings from IOP sensor 108 and/or reservoir sensor 130, patient's calculated IOP, dosage parameters, and/or any other data collected by system 100. The therapeutic dosage parameters may include, but not limited to the factors that determine the frequency and amount of therapeutic agent to be delivered to a patient.

In one example, communication module 112 is an active communication module such as a radio. As an active communication module, data collected by system 100 is actively transmitted to external device 114 positioned external of the patient. In other embodiments, communication module 112 is a passive module. For example, communication module 112 may be a passive RFID device. As such, communication module 112 is operable to transmit and receive data when activated by radio frequency signals to the external device 114.

As discussed above, communication module 112 is operable to transmit and receive data to and from external device 114. For example, external device 114 may include, but not limited to a computer system particularly arranged to communicate with system 100, PDA, cell phone, wrist watch, custom device exclusively for this purpose, remote accessible data storage site (e.g. an internet server, email server, text message server), or other electronic device. As such, these external devices allow a healthcare professional to monitor and treat a patient's eye disorder such as glaucoma.

For example, the healthcare professional can receive data relating to a patient's IOP, pressure in reservoir storage 126, amount of therapeutic agent remaining in reservoir storage 126, and/or any other data collected by system 100 from communication module 112 on external device 114 (e.g. computer). Based upon the received data, the healthcare provider can diagnose and determine whether the dosage parameters stored in system 100 adequately address the patient's needs. If the healthcare provider determines that the dosage parameters need altering or updating, then the healthcare provider can interface with system 100 through communication module 112. In such a scenario, the healthcare provider can alter or update the dosage parameters stored in processor 102 and/or memory 110 via their external device 114 (e.g. computer). Thus, communication module 112 enables the healthcare provider to have an accurate accounting of the patient's eye condition (e.g. IOP condition) as well as the ability to alter the course of treatment if needed.

In some embodiments communication module 112 is operable to receive data transmissions/signals that can be used to charge power source 104. In other words, signals received by communication module 112 can be used to provide energy to system 100, including the ability to charge power source 104. In some examples, communication module 112 includes an antenna capable of harvesting energy through inductive coupling with one of the external devices discussed above. In that regard, communication module 112 can harvest energy from signals, such as radio frequency waves, in order to provide power to system 100.

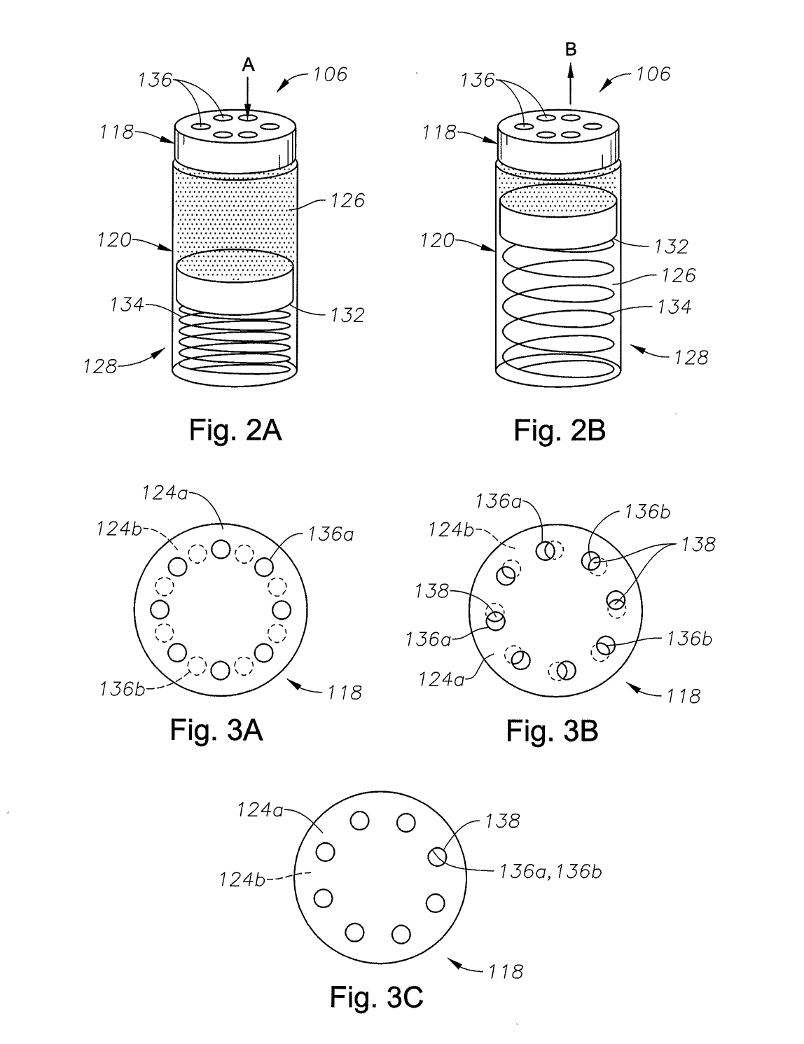

FIGS. 2A and 2B illustrate exemplary dispenser 106 of the implantable variable drug delivery system 100. In particular, FIG. 2A shows dispenser 106 being filled with a therapeutic agent while FIG. 2B shows dispenser 106 dispensing the therapeutic agent. As discussed above, dispenser 106 includes shutter assembly 118 and reservoir assembly 120. As shown, reservoir assembly 120 includes reservoir storage 126 and reservoir actuator 128. Here, reservoir storage 126 stores a therapeutic agent while reservoir actuator 128 applies a pressure against the therapeutic agent. In that regard, reservoir actuator 128 in FIGS. 2A and 2B includes a piston 132, or plate member, that is attached to a biasing member 134. In this example, biasing member 134 is a compressed spring that is forcing piston 132 to exert pressure against the therapeutic agent.

Shutter assembly 118 enables the release of the therapeutic agent from reservoir storage 126. In that regard, shutter assembly 118 controls the movement of one or more irises 136 that allow for the therapeutic agent to be dispensed from dispenser 106. As shown in FIG. 2A by arrow A, shutter assembly 118 is actuated such that irises 136 allow for a therapeutic agent to be delivered into reservoir storage 126 either in vivo or before implantation of dispenser 106. As shown, the potential energy needed by reservoir actuator 128 to later dispense the therapeutic agent is produced by compressing the biasing member 134 upon filling of reservoir storage 126 with the therapeutic agent.

As shown in FIG. 2B by arrow B, shutter assembly 118 is actuated such that irises 136 allow the therapeutic agent to dispense from reservoir storage 126. As shown, the biasing member 134 applies loading against the piston 132, which in turn presses against the therapeutic agent to dispense the agent through shutter assembly 118.

FIGS. 3A-3C are illustrations of shutter assembly 118 of the implantable variable drug delivery system 100 having an active iris to control the flow rate of a therapeutic agent being dispensed by the shutter assembly. As shown, shutter assembly 118 has shutters 124a and 124b with irises 136a and 136b, respectively. In one example, the shutters 124a and 124b are formed of plate members moveable relative to each other. In that regard, either shutter 124a or 124b remains stationary while the other shutter rotates about its axis to either align or non-align irises 136a and 136b of the respective shutters. As the irises 136a and 136b align or overlap, they form a variable-sized through opening (e.g. orifice) referred to herein as the active iris 138. The therapeutic agent stored in reservoir storage 126 is released through active iris 138. In the example shown, the active irises 138 provide multiple delivery paths for administering a therapeutic agent.

In FIG. 3A, irises 136a and 136b are unaligned such that the shutter assembly is closed thereby preventing the release of therapeutic agent from reservoir storage 126. Referencing FIG. 3B, as either shutter 124a or 124b rotates about its axis while the other shutter remains stationary, irises 136a and 136b become partially aligned. As shown, the partial alignment creates active iris 138 that has a cross-sectional area less than the cross sectional area of any one iris 136a or 136b. The active iris 138 allows for more control over the flow rate of the therapeutic agent through shutter assembly 118. Because the partial alignment of irises 136a and 136b provides an opening (e.g. active iris 138) having less cross-sectional area than if the irises were completely aligned (e.g. mirror images shown in FIG. 3C and described below) the amount of therapeutic agent released by shutter assembly 118 is reduced. Thus, the flow rate of therapeutic agent out of reservoir storage 126 is influenced by the degree of alignment irises 136a and 136b with respect to each other.

FIG. 3C shows the irises 136a and 136b completely aligned (e.g. mirror images) with one another such that the cross-sectional area of active iris 138 is substantially equal to the cross-sectional area of any one of iris 136a or 136b. Thus, when irises 136a and 136b are completely aligned (e.g. mirror images) the flow rate out of reservoir storage 126 is at a maximum.

Although FIGS. 3A-3C show shutter assembly 118 having a substantially circular shape, no limitation on the shape of the components comprising the assembly is implied. For example, the components of shutter assembly 118 may have any conceivable shape including, but not limited to elliptical, oval, square, triangle, rectangular, etc. Moreover, the components comprising a particular shutter assembly are in no way limited to having the same shape. For example, the shutters may have a substantially circular shape while the irises have an elliptical shape. Furthermore, the components of a shutter assembly may have different sizes from one another. By way of example, the irises of one shutter may have a larger cross sectional opening than the cross sectional opening of the irises on another shutter.

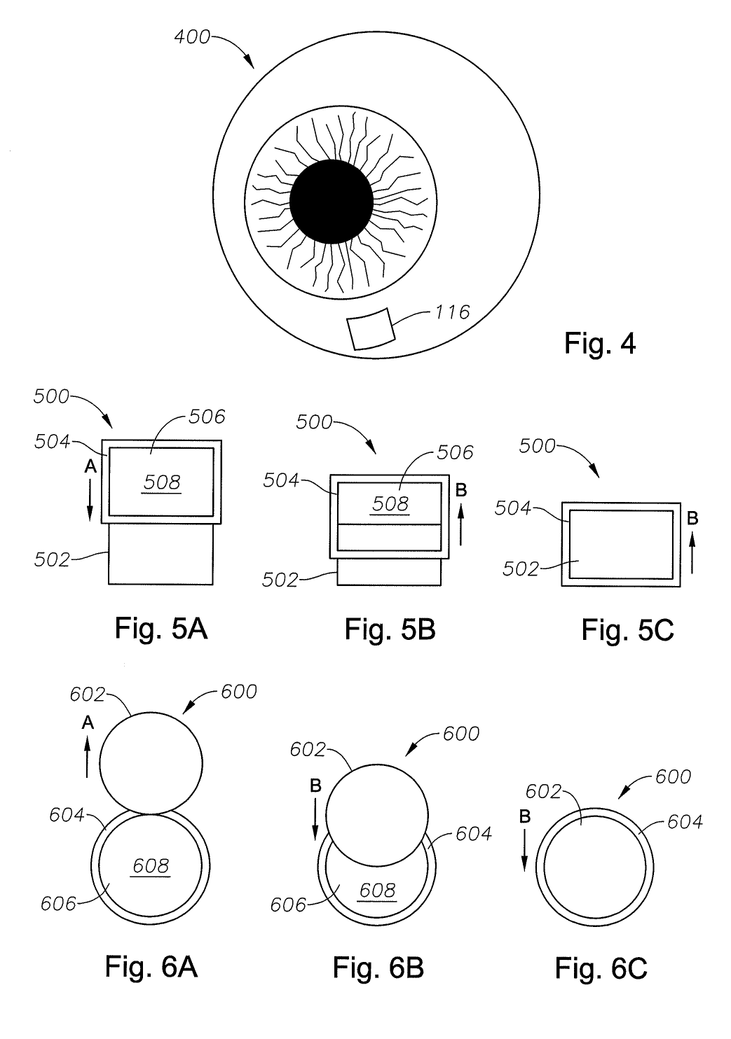

FIG. 4 is an illustration of a perspective view of a patient's eye with an implantable portion 116 of the variable drug delivery system 100 implanted therein. Referencing FIGS. 1 and 4, the exemplary implantable portion 116 includes, but not limited to processor 102, power source 104, dispenser 106, IOP sensor 108, memory 110, and communication module 112. For example, some or all of the components of implantable portion 116 may be implanted under the conjunctiva of eye 400. In other embodiments, however, some or all of the components of implantable portion 116 may be implanted on the exterior of the sclera of eye 500. Moreover, it is contemplated that implantable portion 116 and/or one or more of the components of system 100 can be implanted anywhere within the eye.

FIGS. 5A-5C are illustrations of another exemplary shutter assembly 500 having an active iris to control the flow rate of a therapeutic agent being dispensed by the shutter assembly. The shutter assembly 500 may form part of the dispenser 106 in FIG. 1 any may include the shutter actuator 122. Shutter assembly 500 has a substantially rectangular shape and includes shutters 502 and 504. Here, as shown in FIG. 5A, shutter 504 has an iris 506, or opening, while shutter 502 is void of an iris. In this example, shutter 502 is translated in the direction of arrow A to avoid occluding iris 506. In other examples, shutter 502 is rotated, pivoted, and/or moved to achieve the position of shutter 502 as shown in FIG. 5A. By positioning shutter 502 in the manner shown in FIG. 5A, an active iris 508, or through opening, is created such that the therapeutic agent stored in reservoir storage 126 is released through active iris 508. In this position, flow rate through active iris 508 is at a maximum because the cross-sectional area of active iris 508 is substantially equal to the cross-sectional area of iris 506. Thus, by translating shutter 502 in the direction of arrow A the cross sectional size of through opening 508 increases such that the amount of therapeutic agent released from reservoir storage 126 is increased.

Referring to FIG. 5B, translating shutter 502 in the direction of arrow B allows for more control over the flow rate of the therapeutic agent through shutter assembly 500. As 502 translates in the direction of arrow B, the cross sectional size of active iris 508 decreases such that the amount of therapeutic agent released from reservoir storage 126 is limited in part by the decreasing size of the active iris 508. As shown, active iris 508 has a cross-sectional area that is less than the cross sectional area of iris 506. Because active iris 508 in FIG. 5B has a smaller cross-sectional size than the active iris 508 in FIG. 5A the amount of therapeutic agent released by shutter assembly 500 is reduced. Thus, the flow rate of a therapeutic agent through shutter assembly 500 is influenced by the degree of translation of shutter 502 with respect to shutter 504.

FIG. 5C shows shutter 502 translated in the direction of arrow B to cover iris 506 such that active iris 508 no longer exists (e.g. closed). As shown, shutter 502 is sized and shaped to completely cover iris 506. When shutter 502 completely covers irises 506 the release of therapeutic agent from reservoir storage 126 is prevented. Thus, the release of therapeutic agent from reservoir storage 126 is prevented by the complete covering of iris 506 by shutter 502 when translated in the direction of arrow B.

FIGS. 6A-6C are illustrations of another exemplary shutter assembly 600 having an active iris to control the flow rate of a therapeutic agent being dispensed by the shutter assembly. The shutter assembly 600 may form part of the dispenser 106 in FIG. 1 any may include the shutter actuator 122. Shutter assembly 600 has a substantially circular shape and includes shutters 602 and 604. Here, as shown in FIG. 6A, shutter 604 has an iris 606, or opening, while shutter 602 is void of an iris. In this example, shutter 602 is translated in the direction of arrow A to avoid occluding iris 606. In other examples, shutter 602 is rotated, pivoted, and/or moved to achieve the position of shutter 602 as shown in FIG. 6A. By positioning shutter 602 in the manner shown in FIG. 6A, an active iris 608, or through opening, is created such that the therapeutic agent stored in reservoir storage 126 is released through active iris 608. In this position, flow rate through active iris 608 is at a maximum because the cross-sectional area of active iris 608 is substantially equal to the cross-sectional area of iris 606. Thus, by translating shutter 602 in the direction of arrow A the cross sectional size of through opening 608 increases such that the amount of therapeutic agent released from reservoir storage 126 is increased.

Referring to FIG. 6B, as 602 translates in the direction of arrow B, the cross sectional size of through opening 608 decreases such that the amount of therapeutic agent released from reservoir storage 126 is limited in part by the decreasing size of active iris 608. As shown, active iris 608 has a cross-sectional area that is less than the cross sectional area of iris 606. Translating shutter 602 in the direction of arrow B allows for more control over the flow rate of the therapeutic agent through shutter assembly 600. Because through opening 608 in FIG. 6B has a smaller cross-sectional size than through opening in FIG. 6A the amount of therapeutic agent released by shutter assembly 600 is reduced. Thus, the flow rate of a therapeutic agent through shutter assembly 600 is influenced by the degree of translation of shutter 602 with respect to shutter 604.

FIG. 6C shows shutter 602 translated in the direction of arrow B to cover iris 606 such that active iris 608 no longer exists (e.g. closed). As shown, shutter 602 is sized and shaped to completely cover iris 606. When shutter 604 completely covers irises 606 the release of therapeutic agent from reservoir storage 126 is prevented. Thus, the release of therapeutic agent from reservoir storage 126 is prevented by the complete covering of iris 606 by shutter 602 when translated in the direction of arrow B.

Although FIGS. 5A-5C and FIGS. 6A-6C show exemplary shutter assemblies 500 and 600 having a substantially rectangular or circular shape, respectively, no limitation on the shape of the components comprising these respective assemblies is implied. For example, the components of shutter assemblies 500 and 600 may have any conceivable shape including, but not limited to, elliptical, oval, square, triangle, rectangular, circular, etc. Moreover, the components of a particular shutter assembly are in no way limited to having the same shape. For example, the shutters may have a substantially circular shape while the iris has an elliptical shape. Furthermore, the components of a particular shutter assembly may have different sizes from one another. By way of example, one shutter may cover a larger cross sectional area than another shutter. Additionally, even though shutter assemblies 500 and 600 have been described as two shutters and one iris, respectively, it is contemplated that these shutter assemblies may have one or more shutters and/or one or more irises. Also, in other examples the respective movement or positioning of any one shutter within shutter assemblies 500 and 600 is accomplished by rotating, pivoting, turning and/or otherwise moving the shutter to a desired position.

In addition, it is within the scope of this disclosure that all active irises within a particular shutter assembly may operate in unison or independent of one another. For example, upon actuation of the shutter assembly all active iris may open and/or move towards closure at substantially the same time such that the respective active irises all have substantially the same cross sectional area. In some examples, upon actuation of the shutter assembly only one or more, but not all of the active irises may move open and/or move towards closure such that respective active irises have substantially different cross sectional areas.

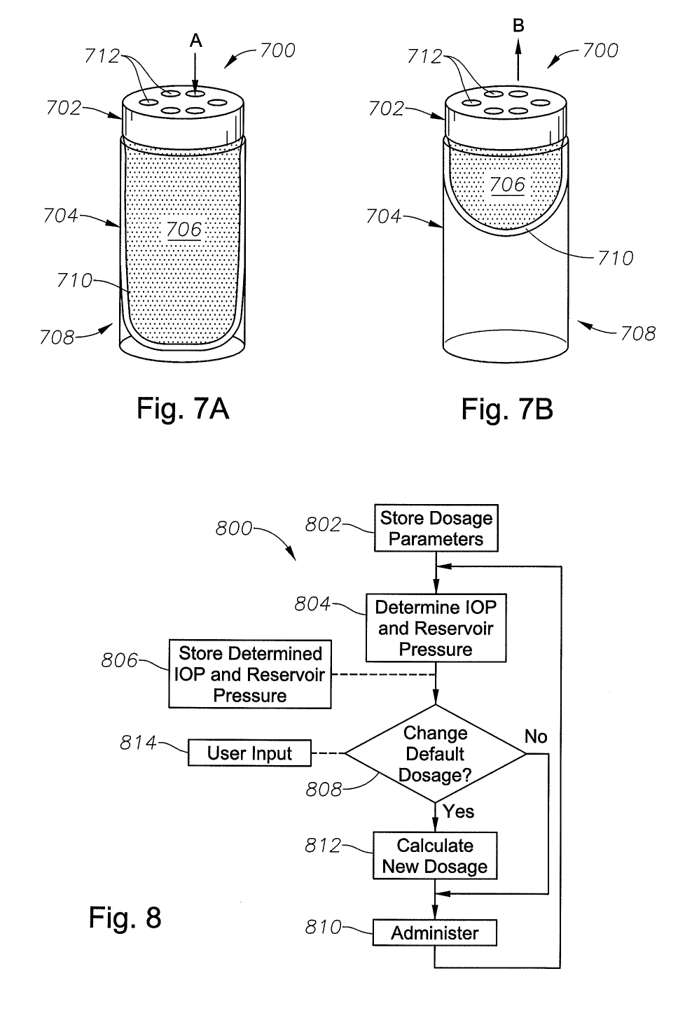

FIGS. 7A and 7B illustrate another exemplary dispenser 700 usable with the implantable variable drug delivery system disclosed herein. In particular, FIG. 7A shows dispenser 700 being filled with a therapeutic agent while FIG. 7B shows dispenser 700 dispensing the therapeutic agent. Dispenser 700 includes shutter assembly 702 and reservoir assembly 704. As shown, reservoir assembly 704 includes reservoir storage 706 and reservoir actuator 708.

Here, reservoir storage 706 stores a therapeutic agent while reservoir actuator 708 applies a pressure against the stored therapeutic agent. In that regard, reservoir actuator 708 includes an elastic member 710, or pouch, that is positioned within reservoir storage 706. For example, elastic member 710 is an elastic pouch having a variable volume. Because of its elastic nature, the member 710 is expandable or stretchable to contain a volume of the therapeutic agent. The stretched elastic is biased towards its unstretched shape, or predetermined shape, causing the therapeutic agent to be pushed towards shutter assembly 702.

In one example, the elastic pouch has an unstretched shape that biases the pouch towards assuming a substantially flat surface adjacent to and/or in contact with the shutter assembly 702. In such a scenario, upon actuation of the shutter assembly 702 the elastic pouch attempts to assume its unstretched shape (e.g. substantially flat surface adjacent to and/or in contact with the shutter assembly 702) and thereby applies pressure against the stored therapeutic agent in the direction of shutter assembly 702. Moreover, as shown in FIG. 7A, by filling reservoir storage 706 with the therapeutic agent the elastic member 710 is forced to expand and/or stretch away from its unstretched shape. However, as shown in FIG. 7B, upon actuation of the shutter assembly 702 to release the stored therapeutic agent, the elastic member 710 is biased towards assuming its unstretched shape.

Shutter assembly 702 enables the release of the therapeutic agent from reservoir storage 706. In that regard, shutter assembly 702 controls the movement of irises 712 that allow for the therapeutic agent to be dispensed from dispenser 700. As shown in FIG. 7A by arrow A, shutter assembly 702 is actuated by the shutter actuator 122 in FIG. 1 such that irises 712 allow for a therapeutic agent to be delivered into reservoir storage 706 either in vivo and/or before implantation of dispenser 700. As shown, the potential energy needed by reservoir actuator 708 is produced by filling reservoir storage 706 with the therapeutic agent. As discussed above, the filling of the therapeutic agent in reservoir storage 706 causes the elastic member 710 to expand and/or stretch away from its unstretched shape. Thus, the force exerted by the elastic member 710 on the stored therapeutic agent to return to its unstretched shape provides the potential energy needed by the reservoir actuator 708 to expel the therapeutic agent from dispenser 700.

As shown in FIG. 7B by arrow B, shutter assembly 702 is actuated such that irises 702 allow for the dispensing of the therapeutic agent from reservoir storage 706. As shown, the elastic member 710 is biased towards returning to its unstretched shape thereby forcing the therapeutic agent towards shutter assembly 702. Thus, the therapeutic agent is dispensed from dispenser 700 by the combination of the shutter assembly 702 and the reservoir assembly 704.

FIG. 8 is an exemplary flow diagram showing steps for determining the drug dosage delivered into the patient's eye using the implantable variable drug delivery system 100. Method 800 begins at step 802 with a step of storing dosage parameters in memory 110 and/or processor 102 of system 100. The dosage parameters represent the logic used by system 100 to determine the dosage to administer to the patient to treat an eye disorder such as glaucoma. The dosage parameters can be stored in memory 110 and/or processor 102 prior to, during, or after implantation of system 100 within the patient's eye.

The dosage parameters represent programming logic that allows processor 102 to determine the frequency, amount, and/or which therapeutic agent to administer to a patient. Moreover, processor 102 is operable to control shutter assembly 118 in order to vary the amount of therapeutic drug to be administered. The specific amount of therapeutic agent administered by system 100 is influenced by the flow rate of the therapeutic agent through shutter assembly 118. Factors considered by system 100 in determining the flow rate may include, but not limited to the reservoir storage pressure measured by reservoir sensor 130 and/or the cross sectional area of an active iris (e.g. active iris 138) for the dispensing of the therapeutic agent there through. As discussed above, the flow rate of a therapeutic agent being dispensed by system 100 in part is based on the pressure in reservoir storage 126 and the size of the cross-sectional area of the active iris created in part by one or more irises of the shutter assembly 118. Thus, system 100 allows for varying the dosage amount of a therapeutic drug by considering the reservoir storage pressure and effectively varying the cross-sectional size of the active iris in order to vary the dosage amount.

In some aspects, the dosage parameter includes a default dosage. The default dosage represents a dosage of a therapeutic agent that is administered to the patient as established by the healthcare provider without accounting for data accumulated by system 100 (e.g. measured IOP by sensor 108 and/or measured reservoir storage pressure). Thus, in some embodiments, system 100 is implemented to administer a default dosage regimen that is not altered after being stored in system 100 regardless of the collected data.

Step 804 represents processor 102 receiving pressure readings from IOP sensor 108 and reservoir sensor 130. Based upon the readings, the processor 102 subsequently determines the patient's IOP and the pressure within the reservoir storage 126. As discussed above, some embodiments of the system store the pressure readings from IOP sensor 108 and reservoir sensor 130. The dashed line at step 806 represents the optional nature of storing the pressure readings of IOP sensor 108 and/or reservoir sensor 130 in memory 110.

The operation of system 100 continues to step 808 where the system determines whether to change the default dosage. As discussed above, the default dosage can be administered to the patient without accounting for and/or considering the data accumulated by system 100 (e.g. measured IOP and/or reservoir storage pressure). If system 100 has been programmed as such, then the system administers the default dosage at step 810.

However at step 808, if the dosage parameters have been programmed to account for data accumulated by the system (e.g. measured IOP and/or reservoir pressure), then processor 102 determines the dosage to administer to the patient based on the collected data. In response to the collected data, processor 102 may change the default dosage.

For example, accessing the dosage parameters, processor 102 can be programmed to compare the patient's measured IOP against an acceptable range for the patient's IOP as set forth in the dosage parameters. If the patient's measured IOP falls outside of the acceptable range of IOP, then processor 102 may change the default dosage. If however, the patient's measured IOP falls within the acceptable range of IOP, then the processor may not change the default dosage and subsequently administer the default dosage at step 810.

If the default dosage should be changed at step 808, then at step 812, processor 102 calculates a new dosage (e.g. change the default dosage). Again, the processor 102 may rely upon dosage parameters stored in system 100 for determining a new frequency, amount, and/or type of therapeutic agent to administer to the patient. As discussed above, system 100 varies the dosage amount of a therapeutic drug by considering the reservoir storage pressure and effectively varying the cross-sectional size of the active iris in order to administer a specific dosage amount. After the new dosage has been calculated, system 100 administers the new dosage at step 810. It does this by actuating the shutter 124 using the shutter actuator 122 to control the active iris 138. The reservoir actuator 128 acts on the therapeutic agent to force the agent through the active iris 138.

In some examples, the step of determining whether to change the default dosage at step 808 includes considering a user input. The dashed line at step 814 represents the optional nature of considering user input. As discussed above, a healthcare provider can interface with system 100 via external device 114. As such, the healthcare provider can alter or update the stored dosage parameters via the communication module 112. Thus, the healthcare provider can instruct system 100 to change the default dosage thereby altering the patient's course of treatment.

Upon administering the default dosage or new dosage at step 810, the method of operation for system 100 returns to step 804. As such, the system continues to monitor and measure the patient's IOP and the reservoir storage pressure until the IOP or other parameters dictate that the system administer another dosage of a therapeutic agent.

In summary, implantable system 100 allows for the monitoring and treating various eye disorders. For example, system 100 allows for monitoring excessive fluctuations of a patient's IOP. Unlike traditional treatments for IOP, patient compliance is a non-issue because the implantable system 100 automatically delivers the therapeutic agent at the appropriate dosage. Moreover, because system 100 has the ability to continuously monitor and store IOP data, the system allows a healthcare provider to access and download a complete overview of the patient's IOP for a given time period. The doctor then has the ability to review this extensive IOP data in order to make a more accurate decision regarding future care of the patient (e.g. alter dosage parameters).

In addition, system 100 allows a healthcare provider to set a default dosage to administer the therapeutic agent to treat the patient's IOP. In that regard, system 100 is operable to release the drug at the default dosage without accounting for data accumulated by system 100 (e.g. measured IOP). Additionally, as discussed above, the system 100 can release the therapeutic agent as determined by a closed loop feedback control system based on IOP. Particularly, system 100 can release the therapeutic agent at a default dosage rate initially for a predetermined amount of time and then can change the dosage amount over to a closed loop control method in which the system uses a closed loop feedback based on IOP measurements to adjust the dosage. Additionally, it is contemplated that system 100 may also have a high and low dosage limit to prevent an over-dosage or under-dosage of a therapeutic agent.

While the present disclosure has been illustrated by the above description of embodiments, and while the embodiments have been described in some detail, it is not the intention of the applicant to restrict or in any way limit the scope of the present disclosure to such detail. Additional advantages and modifications will readily appear to those skilled in the art. Therefore, the present disclosure in its broader aspects is not limited to the specific details, representative apparatus and methods, and illustrative examples shown and described. Accordingly, departures may be made from such details without departing from the spirit or scope of the applicant's general or inventive concept.

We claim:

1. A device implanted into an eye of a patient for treatment of glaucoma, the device comprising:
an implantable dispenser comprising:
an implantable reservoir configured to store a therapeutic agent;
an implantable reservoir sensor configured to measure a pressure within the reservoir; and
an implantable processor coupled to the implantable reservoir sensor and configured to receive the measurement of the pressure within the reservoir and determine a dosage of therapeutic agent based on the measurement of the pressure within the reservoir,
wherein the implantable dispenser is configured to release the dosage of the therapeutic agent at a selectively variable rate from the implantable reservoir into the eye.

2. The device of claim 1, wherein the implantable dispenser includes a shutter assembly having a shutter actuating mechanism and at least one shutter, the shutter actuating mechanism configured to move the at least one shutter to enable the release of the dosage of the therapeutic agent stored in the implantable reservoir.

3. The device of claim 2, wherein the at least one shutter includes a first shutter having at least one first iris and a second shutter having at least one second iris, the first and second shutters being arranged such that rotation of the first shutter aligns the at least one first iris with the at least one second iris to create an active iris to enable the release of the dosage of the therapeutic agent stored in the implantable reservoir.

4. The device of claim 2, wherein the shutter assembly has at least one active iris in communication with the reservoir such that actuating of the shutter actuating mechanism varies a size of a cross sectional area of the active iris to vary an amount of dosage released by the dispenser.

5. The device of claim 2, wherein the implantable dispenser further includes a reservoir actuator disposed within the reservoir that upon actuation moves the therapeutic agent from the reservoir to the shutter assembly.

6. The device of claim 5, wherein the reservoir actuator includes a biasing member coupled to a plate member arranged such that the biasing member biases the plate member towards the shutter assembly in a manner that operates to move the therapeutic agent in the reservoir toward the shutter assembly.

7. The device of claim 5, wherein the reservoir actuator includes a stretchable elastic member containing the therapeutic agent.

8. The device of claim 7, wherein upon actuation of the shutter assembly the elastic member compresses to move the therapeutic agent from the reservoir through the shutter assembly.

9. The device of claim 1, further comprising an implantable intraocular pressure (IOP) sensor configured to measure an IOP of the eye and communicate the IOP to the implantable processor, the IOP sensor being sized for implantation into the eye.

10. The device of claim 9, wherein the implantable processor determines the dosage based upon the intraocular pressure of the eye.

11. A method for delivering a dosage of a therapeutic agent into an eye, the method comprising:
implanting a system into the eye, the system comprising:
an implantable dispenser comprising:
an implantable reservoir configured to store the therapeutic agent;
an implantable reservoir sensor configured to measure a pressure within the implantable reservoir; and
an implantable processor coupled to the implantable reservoir sensor and configured to receive the measurement of the pressure within the implantable reservoir,
wherein the implantable dispenser is configured to release the dosage of the therapeutic agent stored in the implantable reservoir into the eye based on the measurement of the pressure within the implantable reservoir;
measuring the pressure within the implantable reservoir with the implantable reservoir sensor;
determining the dosage of the therapeutic agent with the implantable processor by using the measured pressure within the implantable reservoir to determine the dosage; and
actuating the dispenser to dispense the dosage of the therapeutic agent at a selectively variable rate from the implantable reservoir into the eye in response to a signal from the implantable processor.

12. The method of claim 11,
wherein the dispenser includes a shutter assembly having a shutter actuating mechanism and at least one shutter, the shutter assembly being in fluid communication with the implantable reservoir; and
wherein actuating the dispenser includes actuating the shutter actuating mechanism to move the at least one shutter to enable the release of the dosage of the therapeutic agent stored in the implantable reservoir.

13. The method of claim 12, wherein the at least one shutter includes a first shutter having a first iris and a second shutter having a second iris;
wherein actuating the dispenser includes at least partially aligning the first and second irises with respect to each other by rotating the first shutter about its axis with respect to the second shutter to create an active iris to enable the dispensing of the dosage.

14. The method of claim 13, wherein aligning the first and second irises includes aligning the irises such that the active iris is substantially the same size as one of the first and second irises.

15. The method of claim 14, wherein aligning the first and second irises includes aligning the irises such that the active iris is an opening smaller than an opening of at least one of the first and second irises.

16. The method of claim 12, wherein the shutter has at least one iris defining an active iris that is in fluid communication with the implantable reservoir; and
wherein actuating the shutter actuating mechanism to move the at least one shutter alters the active iris's cross sectional size to vary an amount of the dosage released by the dispenser.

17. The method of claim 12, wherein the at least one shutter includes a first shutter having an iris and a second shutter sized and shaped to cover the first iris; and
wherein actuating the dispenser includes translating the second shutter without rotation with respect to the first shutter to create an active iris to enable the release of the dosage.

18. The method of claim 11, further comprising measuring an intraocular pressure of the eye; and
wherein determining the dosage of the therapeutic agent with the implantable processor includes using the measured intraocular pressure to determine the dosage.

19. The method of claim 11, wherein the implantable dispenser further includes a reservoir actuator disposed within the implantable reservoir; and
wherein actuating the dispenser includes applying pressure with the reservoir actuator to the therapeutic agent to dispense the dosage.

20. A device implanted into an eye of a patient for treatment of glaucoma, the device comprising:
an implantable dispenser comprising:
an implantable reservoir configured to store a therapeutic agent;
an implantable reservoir sensor configured to measure a pressure within the reservoir;
an implantable intraocular pressure sensor configured to measure an intraocular pressure of the eye; and
an implantable processor coupled to the implantable reservoir sensor and configured to receive the measurement of the pressure within the reservoir and the measurement of the intraocular pressure, the implantable processor further configured to determine a dosage of the therapeutic agent based on the measurement of the pressure within the reservoir and the measurement of the intraocular pressure,
wherein the implantable dispenser is configured to release the dosage of the therapeutic agent at a selectively variable rate from the implantable reservoir into the eye.

21. The device of claim 20, wherein the implantable dispenser includes a shutter assembly having a shutter actuating mechanism and at least one shutter, the shutter actuating mechanism being configured to move the at least one shutter to enable the release of the dosage of the therapeutic agent stored in the implantable reservoir.

22. The device of claim 21, wherein the at least one shutter includes a first shutter having at least one iris and a second shutter sized and shaped to cover the at least one iris; and
wherein the shutter assembly has a first position where the second shutter completely covers the iris and a second position in which the second shutter translates with respect to the first shutter to create an active iris to enable the release of the dosage of the therapeutic agent stored in the implantable reservoir.
